(19) United States
(12) Patent Application Publication
(10) Pub. No.: US 2022/0206541 A1
(43) Pub. Date: Jun. 30, 2022

(54) SCREEN PROTECTION COVER FOR A HANDHELD DEVICE WITH INFECTION ALERT CAPABILITY

(71) Applicant: Dale K. Crawford, Las Vegas, NV (US)

(72) Inventor: Dale K. Crawford, Las Vegas, NV (US)

(21) Appl. No.: 17/670,388

(22) Filed: Feb. 11, 2022

Related U.S. Application Data
(60) Provisional application No. 63/149,198, filed on Feb. 12, 2021.

(51) Int. Cl. G06F 1/16; H04B 1/3888; H04M 1/18; H04M 1/04; G01N 33/487; G01N 33/569; G01N 21/78

(52) U.S. Cl. CPC G06F 1/1656; H04B 1/3888; H04M 1/185; H04M 1/04; G01N 2800/50; G01N 33/56983; G01N 21/78; G06F 2200/1634; G01N 2800/26; G01N 33/487

(57) ABSTRACT

The present invention relates to a screen protection cover configurable on a handheld device (E.g., a smartphone). The cover includes a transparent cover body, an adhesive member and a chemical agent(s). The transparent body is configured to cover a display screen of the handheld device. The adhesive member is provided on a rear surface of the cover for removably attaching the cover onto a front surface of the display screen of the handheld device. The chemical agent is adapted to change its physical property to alert the user about a potential infection when one or more biological agents transmitted from the user's body come in contact or in proximity to the cover. The physical property includes a color and/or a smell. The user is required to conduct a medically proven disease diagnosis test upon receiving a visible or sensory alert generated by the cover.

### CROSS-REFERENCE TO RELATED APPLICATIONS

This application claims priority to U.S. Provisional Application No. 63/149,198 filed on Feb. 12, 2021. The content of the above application is incorporated by reference in its entirety.

### TECHNICAL FIELD OF INVENTION

The present invention relates to detection of biological agents such as virus, bacteria, pathogens etc. in a user's body responsible for causing infectious diseases such as Ebola, SARS, COVID-19, etc. More particularly, the present invention relates to a novel screen protection cover configured for a handheld device (E.g., a smartphone) that changes at least one of its physical properties such as odor, color, etc. when the biological agents come in contact or in proximity to a front surface of the screen protection cover in order to alert the user to get a medically proven disease diagnosis test done.

### BACKGROUND

Background description includes information that may be useful in understanding the present invention. It is not an admission that any of the information provided herein is prior art or relevant to the presently claimed invention, or that any publication specifically or implicitly referenced is prior art.

The pandemic diseases have a massive impact on human lives, health and quality of life. Such diseases are challenging healthcare systems and economies worldwide.

Some of the well-known pandemic diseases are Ebola, SARS, COVID-19, etc. The testing of patients infected by pandemic disease is very difficult task. Presently, the detection of virus includes pathogen isolation and identification, serological methods, molecular biology methods. Etiological method is the most traditional detection method. Specificity of the etiological method is strong. Also, the results of the etiological method are true and reliable, but the method has some limitations such as for example, it requires considerable time, requires considerable number of staff engagement and has low sensitivity etc.

In some of the diseases, many infected individuals show no symptoms. Therefore, successful epidemiological surveillance strategies are required to monitor the spread of such diseases in the individuals. Further, the outbreak of new strains should include large-scale screening methods that enable periodic and continuous testing of the general population.

However, testing capacity has been limited, hampering attempts to control the spread of disease. Some of the diseases require reliable nasopharyngeal sampling. The RT-PCR testing is highly invasive and requires both specialized staff and appropriate conditions for the manipulation and transport of the samples to comply with clinical standards and protocols expected by regulatory authorities. Lateral flow device antigen tests are cheaper and accurate when detecting individuals with high infection, however, correct testing requires nasopharyngeal sampling. Therefore, regular large-scale testing is difficult because accurate tests are either too invasive, expensive, or logistically complicated to implement, which make them unviable for the task.

There are certain testing methods that have been disclosed in the public domain to overcome the problems from the above-mentioned methods. Configuration of such testing method requires equipment. The size of the equipment, cost effectiveness of the different components of equipment, additional components required by the equipment are some of the challenges faced by the medical institutions or healthcare professionals. Not only this, some equipments are developed using expensive technologies which are incorporated therein and are not effective and have shorter life span costing a lot towards maintenance of such equipments.

Following are some of the attempts made to develop a cost-effective instant disease testing method.

U.S. Pat. No. 8,988,620B2 discloses liquid crystal-based analyte detection that relates to the field of detection of viruses, and in particular to detection of viruses using a liquid crystal assay format. In the present invention, virus binding in a detection region is identified by changes in liquid crystal orientation caused by virus binding independent orientation caused by any topography associated with the detection region.

US2019040451A1 discloses fully integrated hand-held device to detect specific nucleic acid sequences. The device comprises: an extraction chamber adapted to receive a biological sample, wherein said extraction chamber comprises means to extract and lyse the sample to release nucleic acid; a first amplification chamber in communication with the extraction chamber, wherein said amplification chamber comprises means to trigger nucleic acid amplification of a target nucleic acid sequence to occur; and a detection chamber in communication with the amplification chamber, wherein said detection chamber comprises means to detectably label the target nucleic acid and means to detect a signal associated with labeled target nucleic acid, or a single chamber for amplification, detection and identification of multiple nucleic acid sequences.

CN112839125A discloses handheld mobile equipment protective sleeve with virus killing function and preparation process thereof that relates to a handheld mobile equipment protection sleeve with a virus killing function. The handheld mobile equipment protection sleeve sequentially comprises a virus killing paint layer, a sweat-proof paint layer, a waterproof film layer, an adhesive layer and a substrate plate layer, wherein the antivirus paint layer is prepared from the following components in parts by weight: 90 to 95 parts of nano silver-copper alloy mixed particles.

In the light of foregoing, there is need for detecting biological agents such as virus, bacteria, pathogens etc. responsible for spreading pandemic diseases such as Ebola, SARS, COVID-19, etc. in the user's body that overcomes problems prevalent in the prior art.

### SUMMARY

The present invention relates to a screen protection cover configured for a handheld device (E.g., a smartphone) that changes at least one of its physical properties such as odor, color or a combination thereof when the biological agent(s) come in contact or in proximity to a front surface of the screen protection cover in order to alert the user to get a medically proven disease diagnosis test done.

The present invention provides a screen protection cover for a handheld device with infection alert capability wherein the cover receives the biological agent(s) in the form of saliva droplets transmitted from user's body (particularly mouth) or air molecules transmitted from user's skin or breath during exhalation of the air.

The present invention provides a screen protection cover for a handheld device that shows a high sensitivity compared to the existing infection diagnosis testing tools and methods.

The present invention provides a screen protection cover for a handheld device that makes a display screen of the handheld device a good proxy of the health status of an infected user having the handheld device.

The present invention provides a screen protection cover for a handheld device that can be recognized as novel, non-invasive, cost-effective, and efficient method to identify the infectious diseases in the users.

The present invention provides a screen protection that may be useful in limiting the transmission of the pandemic diseases such as Ebola, SARS, COVID-19 etc.

The present invention provides a screen protection cover for a handheld device that cab be detachably removed from the display screen of the handheld device to allow the user to submit the cover with a sample of transmitted biological agent to the medical facilities for conducting a medically proven disease diagnosis test.

The present invention provides a screen protection cover configured for protecting the display screen of the handheld device from environmental factors (E.g., moisture, heat etc.).

These and other objects and advantages of the present invention will become more apparent as the description proceeds, reference being made to the following detailed description and the appended claims along with the accompanying drawings in which like numerals represent like components.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure. In the figures, similar components and/or features may have the same reference label. Further various components of the same type may be distinguished by following the reference label with a second label that distinguishes among the similar components. If only the first reference label is used in the specification, the description is applicable to any of the similar components having the same reference label irrespective of the second reference label.

FIG. 1 shows an assembled view of a screen protection cover configured on a display screen of a handheld device to alert a user about any potential infection, according to an embodiment of the present invention.

FIG. 2 shows an exploded view of the screen protection cover and the handheld device shown in FIG. 1.

FIG. 3 shows the screen protection cover configured onto the display screen of the handheld device and receiving biological agents from the user's body.

### DETAILED DESCRIPTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. If the specification states a component or feature “may”, “can”, “could”, or “might” be included or have a characteristic, that particular component or feature is not required to be included or have the characteristic.

Exemplary embodiments will now be described more fully hereinafter with reference to the accompanying drawings, in which exemplary embodiments are shown. This disclosure may however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

The present invention relates to a screen protection cover configured for a handheld device (E.g., a smartphone) that changes its physical properties such as odor, color, etc. or any combination thereof when the biological agents transmitted form a user's body come in contact or in proximity to a front surface of the screen protection cover in order to alert the user to get a medically proven disease diagnosis test done. The cover receives the biological agents in the form of saliva droplets transmitted from user's body (preferably mouth) or air molecules transmitted from user's skin or breath during exhalation of the air. The cover shows a high sensitivity compared to the existing infection diagnosis testing methods. The cover makes the display screen of a handheld device a good proxy of the health status of an infected user of the handheld device. The cover is a new, non-invasive, cost-effective, and efficient method to identify the infected user. The cover is useful in limiting the transmission of the pandemic diseases such as Ebola, SARS, COVID-19 etc. The detachability of the cover allows the user to submit the cover with a sample containing biological agent(s) for conducting a medically proven disease diagnosis test. The cover protects the display screen of the handheld device from environmental factors (E.g., moisture, heat etc.).

From herein afterwards the screen protection cover is referred as a cover 100.

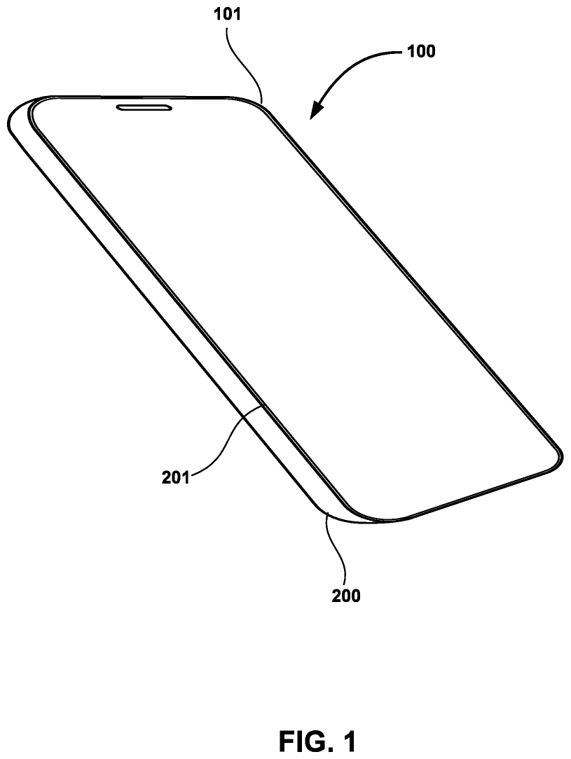

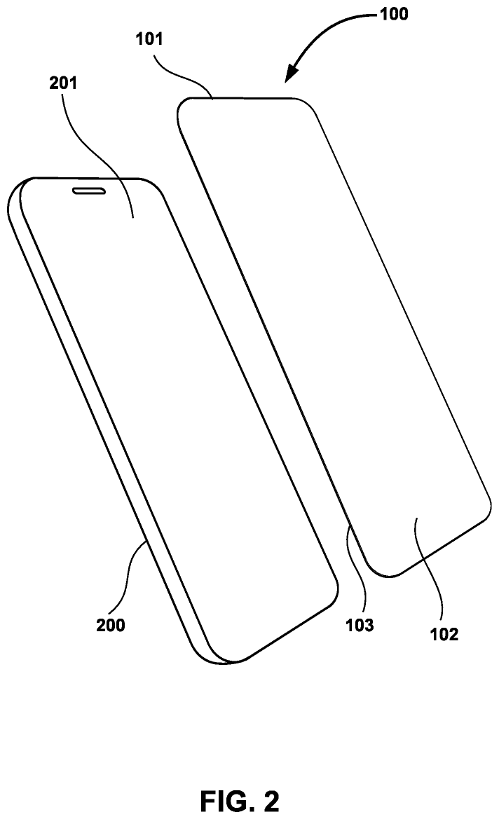

Referring now to FIG. 1 and FIG. 2, the cover 100 includes a transparent body or transparent cover body 101, an adhesive member, and at least one chemical agent.

The transparent body 101 comprises a predefined length, a predefined width and a predefined thickness. The transparent body 101 is configured to cover a display screen 201 of a handheld device 200. The transparent body 101 is sized in length and width in order to substantially cover the display screen of the handheld device 200. The transparent body 101 allows a user of the handheld device 200 to view information displayed on the display screen 201 when the handheld device 200 is operated by the user.

The transparent body 101 is manufactured using plastic, polymer, glass, fiber or any combination thereof or any other suitable material. The transparent body 101 protects the display screen 201 of the handheld device 200 from the environmental factors such as air, moisture, etc. The handheld device 200 may include but not limited to a smartphone, a mobile phone, a tablet, a notepad, a smartwatch or a laptop.

The adhesive member (not seen) is provided on a rear surface 103 of the cover 100. The adhesive member removably attaches the rear surface 103 of the cover 100 onto a front surface 102 of the display screen 201 of the handheld device 200. The adhesive member may include a glue, a binding member, or a sticking member. In a preferred embodiment, the adhesive member is preferably transparent and in a liquified form applied on the rear surface of the cover.

The chemical agent is functionally configured on the transparent body 101 of the cover 100. The chemical agent is adapted to change at least one physical property upon coming in contact with the biological agent (s) transmitted from the infected user. The physical property may include a color and a smell. The physical property (color and/or smell) of the chemical agent changes in a short span of time. In an exemplary non limiting implementation of the present invention, the color and/or the smell of the chemical agent changes within few second to 1 minute. In a preferred embodiment, the chemical agent when changes the color is visibly detectable on the cover 100. The chemical agent(s) that changes the color while coming in contact with the biological agent(s) are metallic ions, salts, precipitations and nonmetallic compounds, etc. In an exemplary non limiting implementation of the present invention, the strontium changes the initial color to the deep red.

The chemical agent that changes the smell while coming in contact with the biological agent(s) are compounds of esters, terpenes, amines, aromatics, aldehydes, alcohols, thiols, ketones, and lactones, etc. Some of the well-known smell changing esters compounds are a geranyl acetate, a fructone, a methyl butyrate and an isoamyl acetate, etc. The well-known smell changing terpenes compounds are a citronellol, a carvone and a eucalyptol etc. The well-known smell changing amines compounds are a trimethylamine, a putrescine, a cadaverine and etc. The well-known smell changing alcohol is a methanol. The well-known smell changing ketones are 6-acetyl-2,3,4,5-tetrahydropyridine and etc. The chemical agent when changes the smell is detectable by sensory cells of a nervous system of the user.

In an alternative embodiment of the present invention, the chemical agent is a chemical compound of the chemical agents in a predefined ratio. The one of the constituent chemical agents is responsible for changing color when comes in contact with the biological agents. Similarly, the one of the constituent chemical agents is responsible for smell changing when comes in contact with the biological agents. Therefore, when such chemical agent comes in contact with the biological agent, the chemical agent changes the color and the smell within the predefined period of time.

In an alternative embodiment of the present invention, the chemical agent capable of changing the size while coming in contact with the biological agent may be used. Some of such chemical agents decreases in size from original size while coming in contact with the biological agent. Similarly, some of such chemical agents increases the size from the original size while coming in contact with the biological agent.

According to the embodiments of the present invention, the chemical agent (s) having properties (color changing or smell detection or size changing) may be either individually used or used in combination over the cover to detect and alert the user about potential infectious diseases that the user may have.

The chemical agent(s) alerts the user about a potential infection when biological agents transmitted from the user's body come in contact or in proximity to the cover 100.

The biological agent(s) may include a virus, a bacterium, an antibody or a pathogen. The biological agent(s) are responsible for one or more infectious disease(s). The biological agents are in the form of saliva droplets transmitted from user's body. Sometimes, the biological agents are in the form of the air molecules transmitted from user while exhaling the air.

In one embodiment, the chemical agent is provided on the front surface 102 of the cover 100. More specifically, the chemical agent is sprayed on the front surface 102 of the cover 100 using a spraying unit (not seen) adapted for storage of the chemical agent. The chemical agent provided on the front surface 102 of the cover 100 lasts for a predefined period of time. In an embodiment, the lifespan of the chemical agent(s) is 1-6 months. In other words, the chemical agent(s) on the cover 100 remains effective for about 1-6 months. The chemical agent(s) can be repeatedly applied on the front surface 102 of the cover 100 after the predefined period of time. The user is required to conduct a medically proven disease diagnosis test upon receiving the alert through changes in the physical property of the chemical agent. The cover 100 can be detached in order to allow the user to submit the cover 100 with a sample containing biological agents received form the user for conducting a medically proven disease diagnosis test. The well-known medically proven disease diagnosis test is RT-PCR, Lateral flow device antigen tests and etc. The medically proven disease diagnosis tests are conducted for conforming the biological agent and the disease.

In another embodiment of the present invention, the transparent body 101 of the cover 100 is manufactured using plastic, polymer, glass and/or fiber together with the at least one chemical agent. The cover 100 is replaceable after the predefined period of time. The user is required to conduct a medically proven disease diagnosis test upon the alert from change in the physical property of the chemical agent. The cover 100 can be detached in order to allow the user to submit the cover 100 with a sample for conducting a medically proven disease diagnosis test.

In yet another embodiment, the front surface of the transparent body 101 may be laminated using a thin transparent film that may embody or made utilizing the chemical agent(s).

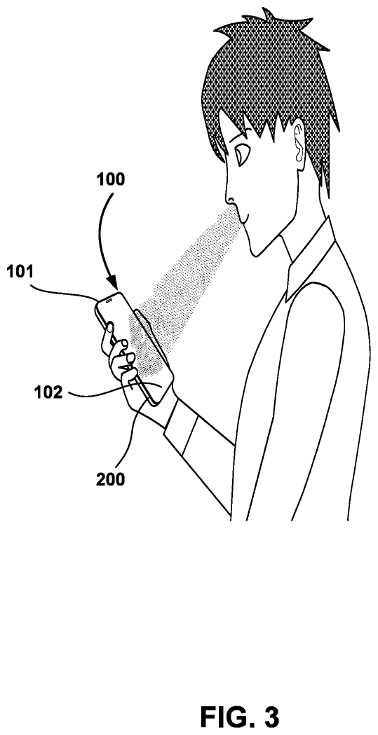

In a given example as shown in FIG. 3, the user's handheld device's display screen 201 is covered using the protective cover 100 comprising the chemical agent (s). During the course of uses of the handled device 200 with the cover 100, if the user is infected by one or more biological agents, the cover upon detecting the traces of such biological agents from the user changes properties of the chemical agent (s). The biological agents are transmitted in the form of saliva droplets from the user's mouth and/or the air molecules from the skin or breath of the user. If the smell or the color or both of these properties of the chemical agent changes leading to change in color and/or smell of the cover 100, the user is required to conduct the medically proven disease diagnosis test. Further, since the cover 100 may be detachably attached on the display screen of the handheld device 200, the user is facilitated to remove the cover 100 for submitting to medical facilities. In this case, the cover acts as a specimen sample (since the cover contains the received biological agent) that may be readily used for conducting the medically proven disease diagnosis test. The cover 100 of the present invention can thus reduce the work load on healthcare system.

The present invention offers an advantage of providing the screen protection cover 100 configured for the handheld device 200 (E.g., a smartphone) that changes its physical properties such as odor, color, etc. when the biological agents come in contact or in proximity to the front surface 102 of the screen protection cover 100 in order to alert the user to get the medically proven disease diagnosis test done. The cover 100 receives the biological agents in the form of saliva droplets transmitted from user's body or air molecules transmitted from user while exhaling the air. The cover 100 shows a high sensitivity compared to the existing infection diagnosis testing. The cover 100 makes the display screen 201 of the handheld device 200 the good proxy of the health status of the user of the handheld device 200. The cover 100 is a new, non-invasive, cost-effective, and efficient method to identify the infected user of the handheld device 200. The cover 100 is useful in limiting the transmission of the pandemic diseases such as Ebola, SARS, COVID-19 etc. The user is allowed to submit the cover 100 with the sample for conducting the medically proven disease diagnosis test. The cover 100 protects the display screen 201 of the handheld device 200 from environmental factors (E.g., moisture, heat etc.).

It should be understood according to the preceding description of the present invention that the same is susceptible to changes, modifications and adaptations, and that the said changes, modifications and adaptations fall within scope of the present invention.

What is claimed is:

1. An infection diagnosing screen protection cover (100) for a handheld device (200), comprising:
a transparent body (101) having a predefined length, a predefined width and a predefined thickness, the transparent body (101) is configured to cover (100) a display screen (201) of a handheld device (200) and allows a user of the handheld device (200) to view information displayed on the display screen (201) when the handheld device (200) is operated;
an adhesive member provided on a rear surface (103) of the cover (100) for removably attaching the rear surface (103) of the cover (100) onto a front surface (102) of the display screen (201) of the handheld device (200);
at least one chemical agent functionally configured on the transparent body (101) of the cover (100), wherein the at least one chemical agent is adapted to change at least one physical property to alert the user about a potential infection when one or more biological agents transmitted from the user's body come in contact or in proximity to the cover (100), wherein, the at least one physical property comprises at least one of a color, and a smell; and
wherein the user is required to conduct a medically proven disease diagnosis test upon the alert from change in the at least one physical property of the at least one chemical agent.

2. The cover (100) claimed in claim 1, wherein the at least one chemical agent is provided on the front surface (102) of the cover (100).

3. The cover (100) claimed in claim 2, wherein the at least one chemical agent provided on the front surface (102) of the cover (100) is effective for a predefined period of time.

4. The cover (100) claimed in claim 2, wherein the at least one chemical agent provided on the front surface (102) of the cover (100) is effective for 1-6 months.

5. The cover (100) claimed in claim 3, wherein the cover (100) is replaceable after the predefined period of time.

6. The cover (100) claimed in claim 3, wherein the at least one chemical agent can be repeatedly applied on front surface (102) of the cover (100) after the predefined period of time.

7. The cover (100) claimed in claim 1, wherein the handheld device (200) comprises at least a smartphone, a mobile phone, a tablet, a notepad, a smartwatch, or a laptop.

8. The cover (100) claimed in claim 1, wherein the adhesive member includes a glue, a binding member, or a sticking member.

9. The cover (100) claimed in claim 1, wherein the one or more biological agents comprises at least a virus, a bacterium, an antibody, or a pathogen.

10. The cover (100) claimed in claim 1, wherein the at least one chemical agent is sprayed on the front surface (102) of the cover (100) using a spraying unit.

11. The cover (100) claimed in claim 1, wherein the cover (100) is detachable in order to allow the user to submit the cover (100) with a sample containing the received biological agent for conducting a medically proven disease diagnosis test.

12. The cover (100) claimed in claim 1, wherein the transparent body (101) of the cover (100) is manufactured using at least a plastic, a polymer, a glass and a fiber.

13. The cover (100) claimed in claim 1, wherein the transparent body (101) of the cover (100) is manufactured using at least a plastic, a polymer, a glass and a fiber together with the at least one chemical agent.

14. The cover (100) claimed in claim 1, wherein the one or more biological agents are in the form of saliva droplets transmitted from user's body or air molecules transmitted from user during exhalation of the air.

15. The cover (100) claimed in claim 1, wherein the adhesive member is transparent and provided in a liquified form applied on the rear surface (103) of the transparent body (101) of the cover (100).
